(19) United States
(12) Patent Application Publication
(10) Pub. No.: US 2004/0185069 A1
(43) Pub. Date: Sep. 23, 2004

(54) Hydroxycitric acid derivatives for body slimming and tone firming compositions

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/394,851

(22) Filed: Mar. 22, 2003

(51) Int. Cl. A61K 31/205; A61K 7/00

(52) U.S. Cl. 424/401; 514/554

(57) ABSTRACT

The present invention discloses cosmetic or topical pharmaceutical compositions for body slimming, firming, cellulite reduction, fat-reduction, and obesity control benefits that can be selective and specific for external body parts and organs such as face, chin, cheeks, arms, “love handles” in abdomen area, eye lids and eye zone, neck, breasts, thighs, and hips. These compositions include a synergistic, bioavailability-enhanced ion-pair combination of Hydroxycitric acid or Hydroxycitric acid derivatives with certain organic bases such as Niacinamide, Niacin, Pyridoxine, Aminophylline, Caffeine, Carnitine, Creatine, Chitosan, Allantoin, Glucosamine, Phaseolamine, Chromium Picolinate, Theobromine, Theophylline, and such.

Certain “Topical Nutraceutical Compositions with Selective Body Slimming and Tone Firming Antiaging Benefits” have been disclosed by the present inventor in U.S. patent application Ser. No. 10/248,508 (filed Jan. 24, 2003).

### BACKGROUND OF THE INVENTION

The enhancement of physical appearance occupies greater focus in human life than nearly all other daily life-related concerns combined. There are far more consumer products available for the beautification of human body than for the treatment of human ailments. The improvement of body tone and appearance is a growing, multibillion-dollar industry encompassing cosmetic, nutraceutical, pharmaceutical, and physical therapy disciplines. The consumer attention is focused on newest miracle ingredient in age-defying, anti-wrinkle, body firming, fat burning, varicose reducing, and lean body mass increasing concoctions, promotions, witchcraft, and claims.

Several new nutraceutical compositions have been disclosed in the prior art for the treatment of obesity and promotion of lean body mass (which is reduction of low specific gravity fatty tissue and increase of higher specific gravity muscle mass).

However, most such compositions are administered as oral dosage forms. Such orally administered compositions have several drawbacks, most notably (1) Their possible unpleasant taste, (2) Their almost certain partial or full degradation during digestion, absorption, and transport into bloodstream, (3) Their further degradation by the oxidizing and reducing enzymes during their transport to various organs of the body where their action is desired, (4) Their lack of selectivity in terms of body part or organ (for example, if the weight loss is selectively desired in the chin, face, or arms area, then such compositions taken orally can not be targeted for such organs selectively), and (5) Their lack of being formulated in synergistic combinations to provide additional beauty enhancing benefits.

While topical delivery systems offer several advantages, including (1) they can be administered at the site where benefits are desired, (2) any unpleasant taste problems are avoided, (3) their loss of activity due to metabolic and enzyme action is minimized, and (4) they can be formulated in synergistic combinations, such topical compositions can also suffer from the disadvantages such as (1) their possible lack of elegant cosmetic skin feel, or being unpleasant in appearance or smell, (2) difficult to formulate in topically bioavailable forms, and (3) their possible instability.

It is the purpose of the present invention to circumvent the problems associated with formulation of topical delivery systems that incorporate compositions that can provide external body part or organ slimming, firming, cellulite reduction, fat-reduction, and obesity control benefits, which can be designed to be selective and specific for external body parts and organs such as face, chin, cheeks, arms, “love handles” in abdomen area, eye lids and eye zone, neck, breasts, thighs, and hips.

Hydroxycitric acid, and a number of organic heteroatom base (organic bases that have at least one nitrogen atom bound in a heterocyclic chemical structure) compositions have received much attention in recent years for obesity control and the management of lean body mass. As mentioned earlier, most such compositions are in the form of oral delivery systems.

U.S. Patent Application No. 20020187943 (Majeed et al.) discloses hydroxycitric acid, obtained from the extracts of Garcinia cambogia plant, to cause a reduction in total body weight and body mass index, a reduction in body fat, an increase in lean body mass, and a reduction in perceived appetite level when taken by oral administration route.

U.S. Pat. Nos. 6,395,296 and 6,160,172 (both to Balasubramanyam et al) relates to a new soluble double metal salt of group IA and II A of (−) hydroxycitric acid, process of preparing the same and its use in beverages and other food products without effecting their flavor and properties. This product with >98% purity can be used safely not only as a food supplement in various nutraceutical formulations and beverages but also for effecting obesity control. These compositions are all orally administered as diet compositions, and not suitable for topical applications.

U.S. Patent Application No. 20020187204 and U.S. Pat. No. 6,413,545 (Alviar et al.) discloses a diet composition for managing body weight including effective amounts of Garcinia cambogia extract (which contains Hydroxycitric acid as its main component), Gymnema sylvestre extract, chromium picolinate, vanadium compound, L-carnitine, and conjugated linoleic acid. The daily effective amounts are administered in three approximately equal doses in conjunction with the daily meals. Although this patent does disclose the application of synergistic combinations of several ingredients for body weight management, all are ingestible compositions useful for a dietary intake and not suitable for topical application. Other derivatives of hydroxycitric acid have also been disclosed, for example U.S. Pat. Nos. 4,028,397; 4,007,208; 4,006,166; 4,005,086; 3,994,927; 3,993,668; 3,993,667; 3,966,772 (all to Guthrie et al.), and U.S. Pat. No. 5,911,992 (Braswell et al.) for fat-burning orally-administered nutraceutical compositions.

Most hydroxycitric acid compositions are based on its alkali or alkaline earth metal salts, such as sodium Hydroxycitrate, potassium Hydroxycitrate, and calcium Hydroxycitrate, and mixtures thereof. This is because Hydroxycitric acid is unstable in its free acid state, and it undergoes a fast chemical cyclization reaction to form tetrahydro-3-hydroxy-5-oxo-furan-2,3-dicarboxylic acid (Garcinia acid). Such cyclized transformation products (i.e. lactones) of Hydroxycitric acid are usually considered to be non-effective in the management and control of obesity, as amply established by Majeed et al. (U.S. Pat. No. 5,783,603). Although such lactones are of poor bioavailability by the oral administration route, their use in topical formulations in a fully bioavailable form, although that would still be surprising and unexpected, has nevertheless not been disclosed in the prior art. Since Garcinia acid is now readily available (U.S. Pat. No. 6,147,228; Ibnusaud), its used in bioavailable topical compositions would offer significant advantages of both higher molar equivalence of hydroxycitric acid and topical bioavailability.

However, as can be appreciated by those versed in the art that such alkali or alkaline earth metal salts of hydroxycitric acid contain much less weight of Hydroxycitric acid itself, on a molar basis. For example, it takes only 208 grams of hydroxycitric acid, but 340 grams of hydroxycitric acid tripotassium salt (which occurs as a monohydrate) to provide one gram mole equivalent of hydroxycitric acid. It would thus be advantageous if hydroxycitric acid itself could be stabilized in any topical compositions to avoid this disadvantage.

U.S. Pat. No. 6,307,080 (Pischel et al.) teaches that salts of pyruvic acid, called pyruvates, have valuable physiological, therapeutic and dietary properties. Pyruvates are used to increase stamina and strength in the sports sector, for weight reduction and body fat reduction. These compositions are all orally administered as diet compositions, and not suitable for topical applications. Pyruvates have structural similarity to Hydroxycitric acid.

Several organic bases have received attention in weight management applications. Niacinamide, carnitine, creatine, chitosan, caffeine, theobromine, and aminophylline are most notable among such bases. These bases, while suitable for oral administration, are poorly absorbed through skin from any topical applications.

U.S. Pat. No. 5,962,482 (Bissett) relates to a method for combating cellulite or reducing localized fatty excesses which comprises administering to a person having cellulite or localized fatty excesses a body slimming amount of a composition containing niacinamide. However, topical formulation of niacinamide compositions has been very difficult. These problems have been discussed by Walling et al. (U.S. Pat. No. 6,455,055) in detail. Walling et al. also reported the need for cosmetic compositions comprising unsolubilized crystalline vitamin B.sub.3 compound(s) and discovered certain cosmetic formulations incorporating the crystalline vitamin B.sub.3 compounds of specific particle size in combination with an emollient. However, such formulations were only applicable to lipstick delivery system, and hence of very limited applicability in other cosmetic products. It is also difficult to obtain niacinamide and niacin that meet the strict particle size specifications mentioned by Walling et al. Although Bissett et al. (U.S. Pat. No. 5,962,482) also disclose certain topical compositions of niacinamide, such compositions contain 12 to 15% niacinamide (in the three examples described in that disclosure) in a carrier base that is very greasy, oily, and of poor absorption into the skin, thus providing poor bioavailability (hence, the use of such high levels of niacinamide in those three examples).

Another nutraceutical ingredient, L-carnitine (an organic base), has found applications for the facilitation of the metabolism of lipids by oral administration route, as disclosed in U.S. Patent Application No. 20020111383 (Hassen).

U.S. Patent Application No. 20020137691 (Murad) claims chromium picolinate, an organic heteroatom base, to provide fat burning efficacy in combination with other synergistic ingredients, which are principally oral pharmaceutical compositions. Although Murad does disclose topical compositions of chromium picolinate in combination with other skin beneficial ingredients, such topical compositions also include sugar derivatives, some of which are not suitable for cosmetically elegant compositions due to their stickiness on skin surface from such topical applications.

U.S. Pat. No. 5,165,935 (Andre et al.) discloses a topical cosmetic composition based on kola seed extract for cellulite reduction. Kola seeds are known to contain high levels of caffeine, an organic heteroatom base.

U.S. Pat. No. 5,658,576 (Soudant) a body slimming composition which contains, as the sole slimming agent, an alpha-tocopherol, benzyl nicotinate, xanthinol or hexyl nicotinate, or alpha-tocopherol acetate. Some of these ingredients, such as benzyl nicotinate and hexyl nicotinate, are organic heteroatom bases.

U.S. patent application No. 20020058075 (D. Jones) claims materials derived from Citrus plants can be administered orally to humans for the purpose of producing or maintaining weight loss as well as for improving the person's physical performance and increasing the person's lean muscle mass. The materials contain at least one of the alkaloids from the group consisting of synephrine, hordenine, octopamine, tyramine and N-methyltyramine. All these alkaloids are organic heteroatom bases.

U.S. Pat. No. 6,433,015 (Meyer) discloses a process for reducing the body weight of overweight humans and domestic animals, such as dogs and cats, by administering creatine, an organic base, is provided. These compositions are all orally administered as diet compositions, and not suitable for topical applications.

U.S. Pat. No. 6,506,420 and U.S. patent application No. 20020137729 (both to Babish) describe a composition for minimizing the absorption of triglycerides and fats in the gastrointestinal tract. The composition comprises psyllium and chitosan (an organic base). U.S. Pat. No. 6,323,189 (Hardinge) describes stable chitosan-containing compositions (especially a stable liquid suspension composition) suitable for a weight treatment program. These compositions are all orally administered as diet compositions, and not suitable for topical applications.

The sports nutrition and weight loss market in the U.S. totaled nearly 10 billion U.S. Dollars in 2001. Of various categories, weight loss segment of the nutraceuticals market posted a whopping 20% growth from 2000 to 2001. The usual weight loss or slimming compositions, which are taken orally, usually have an affect on all body parts, and they can not be made selective or customized to match the biochemistry of just one body part, or a group of body parts. The weight loss, slimming, or excess fat control of external body parts or body organs on a selective basis (such as “double chin”, arms, hips, “love handles”, thigh, cheeks, eye zone, neck, and abdomen), is currently not a well known art. The discovery of such is expected to open a vast new consumer market for such topically beneficial compositions.

Surprisingly, it has now been found in the present invention that HCA and its various derivatives, in combination with certain above mentioned organic bases, form an ion-pair complex. Such complexes provide a dual benefit in a synergistic manner. Since both HCA and its derivatives and the organic bases that are combined by the process of the present invention possess weight loss, slimming, or excess fat control benefits, such compositions can now be devised for weight loss, slimming, or excess fat control of external body parts or body organs on a selective basis (such as “double chin”, arms, hips, “love handles”, thigh, cheeks, eye zone, neck, and abdomen, or other such areas of the body). Moreover, and surprisingly, such compositions are stable and easy to formulate. Also, quite unexpectedly, HCA, even if used in its free acid form, does not cyclize to form lactone derivative. In fact, the lactone derivative (i.e. Garcinia acid) itself can now be used by the method of the present invention to form stable compositions that contain HCA in its acid form, bound in an ion-pair form with an organic base.

### OBJECTS OF THE INVENTION

This invention relates to synergistic, bioavailability-enhanced combinations of certain nutraceutical compositions to provide external body part or organ selective, specific and synergistic topical benefits. This invention further relates to in-situ preparation (by ion-pair delivery system) of the derivatives of Hydroxycitric acid (henceforth called “HCA”) and its analogs with certain organic hetero-atom bases, and their application in topical nutraceutical and cosmetic compositions that provide external body part or organ slimming, body part firming, body part cellulite reduction, body part fat-reduction, and body part obesity control benefits.

It is another objective of the present invention to provide topical delivery systems that offer several advantages, including (1) they can be administered at the site where benefits are desired, (2) any unpleasant taste problems are avoided, (3) their loss of activity due to metabolic and enzyme action is minimized, and (4) they can be formulated in synergistic combinations.

It is yet another objective of the present invention that such topical compositions offer additional advantages such as (1) elegance in their cosmetic skin feel, appearance and smell, (2) ease of formulation in topically bioavailable forms, and (3) stability in use and storage.

### BRIEF DESCRIPTION OF THE INVENTION

I have discovered cosmetic or topical pharmaceutical compositions for body slimming, firming, cellulite reduction, fat-reduction, and obesity control benefits that can be selective and specific for external body parts and organs such as face, chin, cheeks, arms, “love handles” in abdomen area, eye lids and eye zone, neck, breasts, thighs, and hips. These compositions include a synergistic, bioavailability-enhanced ion-pair combination of Hydroxycitric acid or Hydroxycitric acid derivatives with certain organic bases such as niacinamide, pyridoxine, aminophylline, caffeine, carnitine, creatine, chitosan, and such.

### DETAILED DESCRIPTION OF THE INVENTION

Most weight loss dietary compositions, pharmaceuticals, and nutraceutical aids are designed to decrease the amount of body fat in an individual by decreasing the individual's appetite for food, decreasing the amount of food absorption in the individual, slowing down the rate of fatty acid synthesis within the body, or increasing the rate of catabolism of fatty acids. The control of body fat, and various biochemical mechanisms that can be utilized for the management of body fat, control of fatty tissue, maintenance of lean body mass, and slimming of body parts and body organs has been described in detail in U.S. Patent Application No. 20010041708 (to Yuan-Di Chang Halvorsen, et al.), U.S. Pat. No. 5,804,596 (to Majeed et al.), U.S. Patent Application No. 20020187943 (to Majeed et al.), and U.S. Patent Application No. 20020112253 (to S. J. Wakil et al.). The following are some examples of weight loss products and their mechanisms. Dexfenfluramine increases the brain levels of serotonin, a neurotransmitter and neurohormone that quell the appetite. Sibutramine also increases the levels of serotonin, as well as noradrenaline, and works to quell the appetite. Neuropeptide Y inhibitors curb the appetite, as well as stimulating the body to burn more sugars and less fat. Bromeriptine mimics the neurotransmitter dopamine, and may reduce blood sugar and fat production by the liver. Leptin, a hormone generated by adipocytes, affects the hypothalamus. Cholecystokinin, a hormone and neurotransmitter, acts to reduce appetite. Butabindide blocks an enzyme that inactivates cholecystokinin. Orlistat interferes with pancreatic lipase, which results in poor absorption of dietary fat. Insulinotropin is a glucagon-like hormone which prevents obesity by slowing down the emptying of the stomach. Bta-243 stimulates beta-adrenergic receptors on adipocytes, with a resulting increase in the burning of fatty acids. Troglitazone is a synthetic hormone which signals muscle cells to utilize fat for energy, rather than sugars. Cytokine regulators change the activity of hormone-like cytokines and alter the communication among cells, resulting in weight loss. Hydroxycitric acid acts as an inhibitor of enzyme citrate lyase, which subsequently slows down the synthesis of fatty acids and increases the rate at which fatty acids are burned.

The problem with most dietary and nutraceutical compositions is that they are not selective for a body part or organ. Objectionable taste, incomplete absorption from the digestive system, degradation of the active principles present in such compositions by digestive enzymes, and metabolic alteration of those active principles during absorption into the bloodstream and transport to fatty organ are additional problems with such orally administered compositions. These problems are multiplied and magnified when combinations of compositions to achieve complementary therapies or benefits are desired, such as slimming or fat reduction of with antiaging and vascular improvement benefits, slimming of “double chin” with age-spot reduction of face and neck, slimming of thighs and legs with varicose veins reduction, and other such combination benefits. Since orally administered therapies or compositions generally do not provide benefits selective to certain targeted external body parts or organs, the development of topical compositions to achieve such combination benefits for selective application to external body parts or organs is highly desirable. The present invention discloses compositions to achieve such external body part or organ selective combination treatment compositions that provide synergistically enhanced benefits for all combinations of such desirable benefits.

I have discovered a simple method by which derivatives of HCA with organic hetero-atom bases (also referred to as “organic base” henceforth) can be made in-situ for their inclusion in cosmetic or pharmaceutical compositions that provide synergistic external body part or organ slimming, firming, cellulite reduction, fat-reduction, and obesity control benefits. These compositions are made by simple acid-base ion-pair chemical reaction, as shown in Equation 1, between an organic acid (RCOOH) and an organic heteroatom base (R′₃N).

RCOOH+(R′₃N)=[RCOO]⁻+[R′₃NH]⁺ (Equation 1)

The articles of the present invention provide for external body part or organ slimming, body part firming, body part cellulite reduction, body part fat-reduction, and body part obesity control benefits. It is achieved by combining the following three components;

(i) At least one body beneficial composition selected from Hydroxycitric acid, salts of Hydroxycitric acid, and derivatives of Hydroxycitric acid that provides external body part or organ slimming, firming, cellulite reduction, fat-reduction, and obesity control benefits, and

(ii) At least one organic heteroatom base that provides external body part or organ slimming, firming, cellulite reduction, fat-reduction, and obesity control benefits in a synergistic, bioavailability-enhanced manner only in a chemical ion-pair combination with (i), and

(iii) A process of combination of (i) and (ii) above to form a new chemical entity or entities that are more bioavailable and stable, and

(iv) A cosmetically or pharmaceutically acceptable delivery system or carrier base.

Hydroxycitric acid, even if obtained by the acid neutralization of its alkali or alkaline earth metal salts, remains in more bioavailable, uncyclized form when in combination with an organic base. It is postulated, although not proven, that, among the three carboxyl groups that hydroxycitric acid contains in its molecule, the primary carboxyl group (i.e. the carboxyl group attached to a methylene carbon, —CH₂—) is of the strongest acidity. Due to this higher acidity, this primary carboxyl group of hydroxycitric acid binds with organic base in an ion-pair mode (Equation 1). Organic base hydroxycitrate, which is thus formed by this ion-pairing mechanism, is stable and does not cyclize to form Garcinia acid derivative. Moreover, such an organic base ion-pair derivative of hydroxycitric acid is also more bioavailable, and penetrates easily through the skin in topical compositions.

As noted above, niacinamide is an organic heteroatom base that has been disclosed to provide fat-burning benefits. When combined with HCA, this organic base forms a new ion-pair chemical entity, niacinamide hydroxycitrate (as shown in simple terms in Equation 2, although this ion-pair combination is better represented chemically according to Equation 1 in which RCOOH is Hydroxycitric acid and R′₃N is niacinamide, for example).

Niacinamide+Hydroxycitric acid=Niacinamide hydroxycitrate (Equation 2)

As is known in the art, the union of an acid and a base leads to the formation of a salt as part of a neutralization reaction. In the case of diacid and triacid bases, and of dibasic and tribasic acids, the mutual neutralization may vary in degree, producing respectively basic, neutral, or acid salts. A method for synthesizing water-soluble, single component, or multi-component salts of HCA has now been discovered, which includes, for example, reacting HCA (or its alkali and alkaline earth metal salts that have been neutralized with an inorganic acid, or an inorganic acid salt of an organic base, to produce HCA or HCA-Organic base ion-pair in situ) in water with at least one organic base to form a single component salt, or several organic bases to form a multi-component salt, the quantity of organic base or bases depending upon the molecular weight and acidity of organic base or bases to form salts with HCA. The preparation of such salts of HCA has been difficult in the prior art, as amply disclosed by Majeed et al. The salts of HA with organic bases are called the “derivatives of HCA with organic bases” henceforth. This is because such salts are specific chemical entities with their independent physical, chemical, and synergistic biological properties. The term “salts” is confusing to many consumers, as they relate this word to common salt, or sodium chloride. Of course, the derivatives of HCA with organic bases, although technically they are salts, they are not the same as sodium chloride. This is an important aspect that requires careful attention when formulating compositions for the consumer markets. It is also of importance to recognize, once more, that such salts are not mere physical mixtures of two or more different chemical entities that are combined to thus form such salt derivatives.

To illustrate the scope of this invention, Equation 3 shows the formation of ion-pair form of niacinamide hydroxycitrate, a derivative of HCA (an organic acid) with niacinamide (an organic hetero-atom base), in a water solution:

HCA+Niacinamide→Niacinamide Hydroxycitrate (Equation 3)

Additionally, by mixing (in equimolar amounts) an inorganic acid salt of an organic base with a metal salt of HCA, organic base derivatives of HCA can be prepared in-situ, as depicted in Equation 4. This example also illustrates a method by which any HCA derivative, such as sodium HCA or calcium HCA or potassium calcium HCA can be converted into an HCA derivative of an organic base.

Sodium HCA+Niacin hydrochloride+Pyridoxine hydrochloride→Niacin Hydroxycitrate+Pyridoxine Hydroxycitrate+Sodium Chloride (Equation 4).

Multi-component derivatives of HCA with organic bases can also be made by the in-situ method by mixing the reacting components in proportionate molar quantities in water or a mixture of water and water-miscible organic solvent solution, as illustrated in Equation 5.

HCA+Niacinamide+Allantoin+Glucosamine→Niacinamide Hydroxycitrate+Allantoin Hydroxycitrate+Glucosamine Hydroxycitrate (Equation 5)

Novel derivatives of HCA with skin beneficial organic bases can be made by in-situ method of present invention.

The compositions in Equation 1 to 5 can also be made in anhydrous systems by using appropriate water-soluble organic solvent in place of water in the in-situ method. The water-miscible organic solvents include but not limited to glycerin, propylene glycol, butylene glycol, polyethylene glycol, polypropylene glycol, methyl pyrrolidone, pyrrolidone, butylene glycol, hexylene glycol, methylpropanediol, glycol ethers, ethanol, isopropanol, and such. A combination of water and water-miscible organic solvent can also be used for the preparation of HA derivatives with organic bases. The examples shown in Equation 1 to Equation 5 are only illustrative, and they do not represent any limitations of the scope of the present invention.

Although a great number of organic heteroatom bases are available, the selection of an appropriate organic base is made on the basis of the following criteria,

(i) the organic base should have a desirable and complementary body beneficial effect synergistic to the HCA moiety of the HCA-organic base ion-pair derivative,

(ii) the organic base should have a pH less than the physiological pH of human body, when such base is in combination with an HCA,

(iii) the organic base should form a stable derivative in combination with an HCA.

The cosmetically or pharmaceutically acceptable delivery system or carrier base compositions can be in any convenient form, such as a clear liquid, serum, lotion, cream, gel, mask, spray, cleanser, shampoo, shower gel, and conditioner, etc. Such compositions can be pre-made, and other ingredients disclosed in the present invention can be added as a solution in an appropriate solvent, or added directly into such pre-made carrier base compositions. Additionally, such delivery system or carrier base compositions can also be made in situ, as illustrated in several examples provided in this invention. Moreover, the ingredients of the present invention can also be added to already formulated products. For example, in a skin lotion that has already been made, the ingredients for cellulite reduction, antiaging benefits, and collagen and elastase promoters can all be added in the amounts specified in the present invention to provide added benefits from such lotion product. As can be seen from this description, the present invention also provides several convenient methods for manufacturing the compositions of present invention.

It is to be further explained that the quantities of any ingredients and compositions of the present invention are not limited to any specific amounts. The quantities shall be based on safe and effective amounts of such ingredients and compositions, since the ingredients and compositions of the present invention represent a very large class of materials that are cosmetic, nutraceutical, or pharmaceutical groups that have a very widely distributed safety and efficacy range for their various efficacies. It is thus nearly impossible to establish any specific limits on the quantities of such ingredients, especially when such ingredients are used in compositions of synergistic combinations. It is to be noted from various examples of the present invention that it is easily possible to include various compositions for combination benefits in amounts from 0.0001% to in excess of 40%. This is because of current popular consumer trend for such amounts. It is to be further noted that such amounts are not thus assumed to be limited to about 40%, since in future the consumer trend may require higher amounts of such compositions. In future, such amounts may depend on several factors that include safety, efficacy, bioavailability, cost, performance, consumer appeal, competition, stability, and such. Since such conditions are not within the control of present inventor, it is preferred that any amounts of such beneficial combination compositions are not artificially limited to certain specific number or a range of numbers.

### EXAMPLES

The following examples are presented to illustrate presently preferred practice thereof. As illustrations they are not intended to limit the scope of the invention. All quantities are in weight %.

### Example 1

Preparation of Hydroxycitric Acid from HCA Salts.

(1) Tri-potassium Hydroxycitrate Hydrate 34.0

(2) Deionized water 36.0

(3) Hydrochloric Acid (10 molar solution in water) 30.0

Procedure. Mix (1) and (2) with heating to a clear solution. Add (3) and mix. Upon cooling, potassium chloride precipitates out, which is then removed by filtration. A solution of hydroxycitric acid (20%) in water is thus obtained. This solution is useful for other compositions that may require hydroxycitric acid.

### Example 2

In-Situ Preparation of Niacin Dipotassium Hydroxycitrate from Niacin Hydrochloride and Tripotassium Hydroxycitrate.

(1) Tripotassium Hydroxycitrate Hydrate 3.4

(2) Deionized Water 95.3

(3) Niacin Hydrochloride 1.3

Procedure. Mix (1) to (3). Niacin Dipotassium Hydroxycitrate (3.96%) is formed in situ.

Potassium chloride, also formed in this reaction, is precipitated by the addition of ethanol, followed by filtration.

### Example 3

Chitosan Hydroxycitrate and Niacinamide Hydroxycitrate Facial Mask Composition Made by the In-situ combination of Chitosan and Niacinamide with HCA, Respectively.

| | |
|---|---|
| (1) Chitosan | 5.0 |
| (2) Glycerin | 18.0 |
| (3) Water | 69.0 |
| (4) Hydroxycitric acid | 5.0 |
| (5) Niacinamide | 2.5 |
| (6) Preservatives | 0.5 |

Procedure: Mix 1, 2, and 3 to a paste.

Mix 4 to 6 separately to a clear solution. Add this to main batch and mix.

A clear gel product is obtained. It is applied on the face and neck and left for 10 to 30 minutes, then rinsed off.

### Example 4

Niacinamide Hydroxycitrate Serum Made by the In-situ Combination of Niacinamide with HCA.

| | |
|---|---|
| (1) Cognis Hydagen CMF (Chitosan & water) | 50.0 |
| (2) Hydroxycitric acid | 5.0 |
| (3) Coleus forskohlii extract, water soluble | 2.0 |
| (4) Deionized Water | 27.5 |
| (5) Glycerin | 10.0 |
| (6) Niacinamide | 5.0 |
| (7) Preservative | 0.5 |

Procedure. Mix all ingredients together. A clear serum is obtained, with syrupy appearance.

### Example 5

Foaming Facial Cleanser with Creatine Hydroxycitrate Made by In-situ Process from Creatine and Hydroxycitric Acid.

| | |
|---|---|
| (1) Cocamidopropyl betaine | 18.0 |
| (2) Water | 49.5 |
| (3) Sodium Cocoyl Isethionate | 20.0 |
| (4) Polydimethylsiloxane | 2.0 |
| (5) Creatine | 5.0 |
| (6) Hydroxycitric acid | 5.0 |
| (7) Preservatives | 0.5 |

Procedure: Mix 1, 2, and 3 to a paste.

Mix 4 to 7 separately to a clear solution. Add this to main batch and mix.

A cream-like product is obtained.

### Example 6

An Antioxidant Cream Composition with Phaseolamin Hydroxycitrate, Sodium Pyruvate, and Creatine Hydroxycitrate Made by the In-situ Process Combining Phaseolamin and Creatine with HCA.

| | |
|---|---|
| (1) Deionized water | 76.45 |
| (2) Cetearyl alcohol (and) dicetyl phosphate (and) | 5.0 |
| () Ceteth-10 phosphate | |
| (3) Cetyl alcohol | 2.0 |
| (4) Glyceryl stearate (and) PEG-100 stearate 4 | |
| (5) Caprylic/capric triglyceride | 5.0 |
| (6) Rosmarinic acid | 0.1 |
| (7) Tetrahydrodiferuloylmethane | 0.1 |
| (8) Glutathione | 0.1 |
| (9) Diosmin | 0.05 |
| (10) Resveratrol | 0.05 |
| (10) Andrographolide | 0.05 |
| (12) Hesperetin | 0.05 |
| (13) Mangiferin | 0.05 |
| (14) Fragrance | 0.5 |
| (15) Preservatives | 0.5 |
| (16) Hydroxycitric acid | 4.0 |
| (17) Creatine | 1.5 |
| (18) Sodium Pyruvate | 2.0 |
| (19) Phaseolamin | 2.5 |

Procedure. Mix 1 to 5 and heat to 75-80° C. Adjust pH to 4.0-4.5. Cool to 35-40° C. with mixing and homogenize. Add 6 to 19 with mixing. Adjust pH to 4.0-4.5, if necessary.

White to off-white cream

### Example 7

Skin Lotion with Chromium Picolinate Hydroxycitrate and Carnitine Hydroxycitrafe Made by the In-situ process.

| | |
|---|---|
| (1) Water | 74.2997 |
| (2) Mineral Oil | 1.0 |
| (3) Phenoxyethanol | 0.9 |
| (4) Glycerin | 3.8 |
| (5) Deodorized Jojoba Oil | 0.0001 |
| (6) Aloe Vera | 0.0001 |
| (7) Panthenol | 0.0001 |
| (8) Methyl Paraben | 0.2 |
| (9) Propyl Paraben | 0.1 |
| (10) PGMS-SE | 2.0 |
| (11) Stearic Acid | 3.0 |
| (12) Cetyl Alcohol | 1.2 |
| (13) Fragrance | 0.5 |
| (14) Botanical Extract | 0.5 |
| (15) Deionized Water | 5.0 |
| (16) Chromium Picolinate | 0.5 |
| (17) Tripotassium Hydroxycitrate | 3.4 |
| (18) Carnitine Hydrochloride | 1.5 |
| (19) Glutathione | 0.1 |
| (20) Vitamin E Acetate | 2.0 |

Procedure. Skin lotion base was first obtained by mixing ingredients 1 to 12 together, then heating at 70 to 80C for one hour, then cooling to ambient temperature with mixing. A white lotion was obtained. Ingredients 15 to 19 were then mixed separately to a clear solution, which was then added to lotion base. All remaining ingredients were then added and the product mixed to a white lotion composition.

### Example 8

Face & Body Cleanser with Carnitine Hydroxycitrate and Niacinamide Hydroxycitrate Made by the In-situ process.

| | |
|---|---|
| (1) Water | 60.94 |
| (2) Germall II (preservative) | 0.1 |
| (3) Kathon CG (preservative) | 0.06 |
| (4) Sodium Lauryl Sulfate | 18.0 |
| (5) Cocamidopropyl betaine | 10.0 |
| (6) Hydroxy Citric Acid | 4.5 |
| (7) Carnitine | 2.5 |
| (8) Ellagic acid | 0.5 |
| (9) Niacinamide | 2.5 |
| (10) Fructose-1,6-diphosphate | 0.2 |
| (11) Fragrance | 0.5 |
| (12) Botanical Extracts | 0.2 |

Procedure. All ingredients were mixed in a tank. A light yellow clear liquid product was obtained.

### Example 9

The preparation of a 35.0% Niacinamide Hydroxycitrate Serum Obtained from Garcinia Acid (the lactone form of Hydroxycitric acid). (The lactone form opens and converts into the open chain form of hydroxycitric acid by this process in-situ.)

| | |
|---|---|
| (1) Carbowax (PEG-6) | 30.0 |
| (2) Deionized Water | 36.8 |
| (3) Garcinia acid | 19.0 |
| (4) Niacinamide | 12.2 |
| (5) Creatine | 2.0 |

Process: Mix all ingredients till a clear solution is obtained.

### Example 10

Facial Cleanser Composition with Niacinamide Hydroxycitrate (16.5%, by In-situ process) for Facial Fat Reduction.

| | |
|---|---|
| (1) Glycerin | 38.3 |
| (2) Methyl paraben | 0.2 |
| (3) Hydroxycitric acid | 10.4 |
| (4) Niacinamide | 6.1 |
| (5) Deionized Water | 18.5 |
| (6) Phenoxyethanol | 0.9 |
| (7) Sodium Cocoyl Isethionate | 20.0 |
| (8) Sodium Methyl Cocoyl Taurate | 5.0 |
| (9) Actiplex 2789 (Extract of various plants) | 0.1 |
| (10) Fragrance | 0.5 |

Procedure: Mix deionized water, HCA, and niacinamide in a tank separately. A clear solution is obtained. All of the other ingredients are then added, except fragrance, and the mixture is heated and stirred at 60 to 70 degrees C. for about five to ten minutes until the mixture is homogenous. The homogeneous mixture is cooled to room temperature, and fragrance is added with mixing. A paste-like product is formed.

I claim:

1. A synergistic cosmetic or pharmaceutical composition for external body part or organ slimming, firming, cellulite reduction, fat-reduction, and obesity control benefits comprising:
(i) At least one body beneficial composition selected from Hydroxycitric acid (HCA), inorganic metal salts of Hydroxycitric acid, derivatives of Hydroxycitric acid, Hydroxycitric acid esters, Hydroxycitric acid amides, Garcinia acid, and combinations thereof, and
(ii) At least one synergistic organic base, or an acid salt of an organic base, and
(iii) A process for the combination of (i) and (ii) above to form an acid-base ion-pair synergistic chemical combination entity, and
(iv) A cosmetically or pharmaceutically acceptable delivery system or carrier base.

2. A synergistic cosmetic or pharmaceutical composition for external body part or organ slimming, firming, cellulite reduction, fat-reduction, and obesity control benefits comprising:
(i) At least one body beneficial composition selected from Hydroxycitric acid (HCA), inorganic metal salts of Hydroxycitric acid, derivatives of Hydroxycitric acid, Hydroxycitric acid esters, Hydroxycitric acid amides, Garcinia acid, and combinations thereof, and
(ii) A process for the in-situ conversion of (i) to the active acid-forms of HCA, and
(iii) A cosmetically or pharmaceutically acceptable delivery system or carrier base.

3. A composition according to claim 1, wherein the HCA ion-pair derivative can be prepared by an in-situ method from a combination of HCA or its derivative(s) with an organic base, or its derivative.

4. A composition according to claim 1, wherein the HCA ion-pair chemical combination entity can be selected from Allantoin HCA, Glucosamine HCA, Creatine HCA, Carnitine HCA, Niacinamide HCA, Pyridoxine HCA, Chitosan HCA, Niacin HCA, Benzyl Niacin HCA, Methyl Niacin HCA, Caffeine HCA, Aminophylline HCA, Chromium picolinate HCA, Phaseolamin HCA, Theophylline HCA, Theobromine HCA, Synephrine HCA, Hordenine HCA, Octopamine HCA, Tyramine HCA, N-Methyltyramine HCA, and combinations thereof.

5. A composition according to claim 1, wherein the HCA ion-pair chemical combination entity can be from 0.0001% to 60% of the total composition.

6. A composition according to claim 1, wherein the cosmetically acceptable delivery system is selected from a lotion, cream, shampoo, shower gel, cleanser, bath oil, salve, paste, lip balm, serum, gel, body splash, cologne, liposomes, mask, mud, and other such well known topical cosmetic and pharmaceutical delivery systems.

7. The compositions according to claim 1, wherein the cosmetically acceptable delivery system can be traditional water and oil emulsions, suspensions, solutions, gels, colloids, and anhydrous systems, and combinations thereof.

8. A composition according to claim 1, wherein additional skin beneficial ingredients, such as anti-oxidants, surfactants, cleansing agents, bleaching agents, vitamins, hormones, minerals, plant extracts, skin whitening agents, anti-inflammatory agents, concentrates of plant extracts, emollients, moisturizers, skin protectants, humectants, silicones, skin soothing ingredients, sun screens, analgesics, anesthetics, colorants, perfumes, and like can be added to the formulation. The quantities of such ingredients can be as needed, and not limited to any specific amounts.
